(19) United States
(12) Patent Application Publication
(10) Pub. No.: US 2012/0108867 A1
(43) Pub. Date: May 3, 2012

(54) Process for the Production of Purified Xylene Isomers

(76) Inventors: Dana Lynn Pilliod, League City, TX (US); John Di-Yi Ou, Houston, TX (US)

(21) Appl. No.: 13/279,645

(22) Filed: Oct. 24, 2011

Related U.S. Application Data
(60) Provisional application No. 61/408,081, filed on Oct. 29, 2010.

(51) Int. Cl. C07C 5/27; B01J 19/00

(52) U.S. Cl. 585/300; 422/187

(57) ABSTRACT

The invention is directed to a process to produce paraxylene and orthoxylene, including reducing the amount of isomerate recycle from vapor phase xylenes isomerization by providing a parallel configuration of vapor phase and liquid phase isomerization units.

### PRIORITY CLAIM

This application claims the benefit of Provisional Application No. 61/408,081, filed Oct. 29, 2010.

### FIELD OF THE INVENTION

The invention relates generally to the production of paraxylene and orthoxylene, including xylene isomerization, and also to an apparatus for the practice of said process.

### BACKGROUND OF THE INVENTION

The xylene isomers are important intermediates, which find wide and varied application in chemical syntheses. By way of example, paraxylene (PX) is a feedstock for terephthalic acid which finds use in the manufacture of synthetic fibers; metaxylene (MX) is used in the manufacture of dyes, and orthoxylene (OX) is used as a feedstock for phthalic anhydride, which finds use in the manufacture of plasticizers.

Xylenes are found in various fractions, such as coal tar distillate, petroleum reformates, and pyrolysis liquids in admixture with other compounds of like boiling point. The aromatic components are readily separated from non-aromatics by methods such as solvent extraction. A fraction may then be obtained readily, such as by distillation, consisting essentially of C8 aromatics. By “C8 aromatics” is meant aromatic hydrocarbons having 8 carbon atoms, including particularly ethylbenzene and the xylene isomers paraxylene (p-xylene or PX), orthoxylene (o-xylene or OX), and metaxylene (m-xylene or MX).

While difficult to separate due to their similar chemical structures and physical properties and identical molecular weights, there are various methods used to separate the C8 isomers, for instance OX is separable from other C8 aromatics by fractional distillation, and PX is separable by fractional crystallization or selective sorption. Present demand is largely for PX, however, there is a sufficiently large demand for OX that a system maximizing recovering both of these isomers in an energy efficient manner, is highly sought after. At ordinary temperatures at which xylenes are processed in a typical petrochemical plant, the thermodynamic equilibrium content is approximately 24 mol % PX, 56 mol % MX, and 20 mol % OX, based on the total amount of xylenes in said feed.

The production of paraxylene and orthoxylene in a conventional aromatics complex is energy intensive. This is due in part to the significant amount of recycle that is reprocessed through orthoxylene and C9+ aromatics removal. A typical commercial process is illustrated in FIG. 1.

The feed streams to the system comprise C8+ aromatics and may come from one or more sources, including C8+ reformate 1 (see, for instance, U.S. Pat. No. 7,179,367), C8+ Selective Toluene Disproportionation Product 17 (see, for instance, U.S. patent application Ser. No. 12/042,433 now granted as U.S. Pat. No. 7,989,672), C8+ transalkylation product 2 (see, for instance, U.S. Pat. No. 7,663,010), C8+ toluene disproportionation product 15 (see, for instance, U.S. Pat. No. 6,198,013), and any other streams that contain C8 aromatics, such as products from toluene methylation with methanol (see, for instance, U.S. application Ser. No. 12/894,778 now published as 2011/0092755). These streams typically comprise C8 and heavier aromatics which are processed along with a recycle stream 10 in one or more fractionators 16 in the removal of OX and C9+ aromatics in stream 3, which can be subsequently separated in fractionator 14 into OX overhead 4 and C9+ bottoms product 5. The C9 and heavier aromatics (C9+) could have an adverse effect on downstream Paraxylene Recovery 12 and vapor phase xylenes isomerization unit 13 if not removed from the feed stream(s) as bottoms product by the aforementioned fractionation unit 14.

The removal of OX and C9+ aromatics in fractionator(s) 16 thus yields an overhead of C8 aromatics stream 6 which typically contains between 10 and 95 wt % paraxylene. The C8 aromatics stream 6 is processed to selectively recover paraxylene by one or both of selective adsorption or crystallization which is shown as PX recovery 12. The PX product stream 7, typically having 99.7+ wt % paraxylene is recovered, and the balance of C8 aromatics stream 8 passes to vapor phase xylenes isomerization 13. Optionally, in the presence of hydrogen in stream 9, vapor phase xylenes isomerization 13 establishes a near-equilibrium balance of xylene isomers in stream 19 using one or more of a variety of catalysts, per se well-known in the art, which may also convert ethylbenzene to benzene and ethane or may convert ethylbenzene to near-equilibrium xylene isomers. The xylenes isomerization product stream 19 passes to detoluenization fractionation 18 which removes C7 and lighter materials in stream 11 to yield isomerate recycle stream 10. Isomerate recycle stream 10 is processed in the OX and C9+ aromatics removal unit 16.

Improving such an energy-intensive processes is an active area of research, but it is not a simple matter of optimization of each individual step, as optimization of one step may negatively effect one or more steps in the overall system. Examples of proposed improvements include the following.

U.S. Pat. No. 3,856,874 describes splitting the effluent stream from PX separation, passing the independent streams over different catalysts, then combining the isomerized streams and recycling.

U.S. Pat. No. 7,439,412 teaches a process for recovering one or more high purity xylene isomers from a C8+ aromatic feedstream including the use of an isomerization unit under liquid phase conditions. In an example, the product of the liquid phase isomerization unit is returned to the first fractionation tower in the system. See also U.S. Pat. No. 7,626,065.

U.S. Pat. No. 7,553,998 teaches a process for recovering one or more high-purity xylene isomers from a feed having substantial content of C9+ aromatic hydrocarbons comprising de-ethylation of heavy aromatics followed by fractionation and then passing the stream to a C8 aromatic isomer recovery to recover high-purity xylene isomer with lowered energy costs. Streams passing through an isomerization unit under liquid isomerization conditions are split, with a portion sent to an isomer recovery unit, and a portion is purged.

U.S. application Ser. No. 12/612,007 (published as 2010/0152508) describes a process for producing a PX-rich product, the process comprising: (a) providing a PX-depleted stream; (b) isomerizing at least a portion of the PX-depleted stream to produce an isomerized stream having a PX concentration greater than the PX-depleted stream, a benzene concentration of less than 1,000 ppm, and a C9+ hydrocarbons concentration of less than 5,000 ppm; and (c) separating the isomerized stream by selective adsorption.

Provisional Application No. 61/326,445, filed Apr. 21, 2010, is directed to a xylenes isomerization process, including a liquid phase isomerization, for the production of equilibrium or near-equilibrium xylenes, wherein the process conditions include a temperature of less than 295° C. and a pressure sufficient to maintain the xylenes in liquid phase that uses at most only ppm levels of hydrogen and that in embodiments can be regenerated numerous times by a very simple in situ procedure.

Other references of interest include U.S. Publication Nos. 2008/0262282; 2009/0149686; 2009/0182182; U.S. Pat. Nos. 6,448,459; 6,872,866; and 7,368,620.

The present inventors have surprisingly discovered a process which significantly reduces the energy required to produce high purity xylene isomers by providing parallel configuration of vapor phase and liquid phase isomerization systems.

### SUMMARY OF THE INVENTION

The invention is directed to a process for producing paraxylene comprising first separating a feed comprising C8+ aromatics into an overhead, or first stream comprising PX and MX and a bottoms product, or second stream comprising OX and C9+ aromatics, separating the PX and MX stream in a PX recovery unit to recover a PX-rich stream and a PX-depleted stream, then separating said PX depleted (C8 aromatics) stream through a parallel configuration of vapor phase xylenes isomerization and liquid phase xylenes isomerization. The OX and C9+ aromatics stream may then be separated downstream of the first separating step, such as by fractionation.

In embodiments, a benzene separation step occurs between the first fractionation and the PX recovery unit, and/or a benzene separation step downstream from the isomerization step(s). There may also be, in embodiments, a toluene separation step, such as downstream of said isomerization step(s).

In embodiments, the liquid phase isomerization product is recycled to one or more of the first fractionation step, the benzene separation step (where present), and the PX recovery step.

The invention also relates to an apparatus for the production of paraxylene comprising a first fractionation column operating at conditions suitable for the separation of a C8+ aromatics stream into an overheads comprising PX and MX, and a bottoms product comprising OX and C9+ aromatics. The overheads stream fluidly connected with a PX recovery unit, wherein said PX recovery unit provides a PX-enriched stream, a PX-depleted stream, and the bottoms product stream is fluidly connected with an OX/C9+ separation step. The improvement comprising dividing a conduit carrying said PX-depleted stream so that a portion of said PX-depleted stream is passed to a vapor phase isomerization unit, and another portion of said PX-depleted stream is passed to a liquid phase isomerization unit.

In embodiments, said liquid phase isomerization unit is fluidly connected so as to provide liquid phase isomerate recycle to said first fractionation column and/or to said PX recovery unit.

In embodiments, said PX recovery unit is selected from at least one of a crystallizer and an adsorptive separator.

In embodiments, at least one other fractionator upstream of said first fractionator, wherein said at least one other fractionator operates under conditions suitable for removing benzene from a stream comprising xylenes or for removing toluene from a stream comprising xylenes, and optionally wherein both said fractionator for removing benzene and said fractionator for removing toluene are provided upstream of said first fractionator.

It is an object of the invention to significantly reduce the energy required to produce paraxylene and orthoxylene, by minimizing the amount of isomerate recycle from vapor phase xylenes isomerization.

These and other objects, features, and advantages will become apparent as reference is made to the following detailed description, preferred embodiments, examples, and appended claims.

### BRIEF DESCRIPTION OF THE DRAWINGS

In the accompanying drawings, like reference numerals are used to denote like parts throughout the several views.

FIG. 1 is a schematic illustrating typical commercial processing of C8+ aromatics to produce paraxylene.

FIG. 2 is a schematic illustrating an embodiment of the invention.

FIGS. 3 and 4 represent a comparison of two systems, the former returning liquid isomerization product to the rerun tower and the latter returning liquid isomerization product to PX recovery unit.

### DETAILED DESCRIPTION

According to the invention, a system is provided having parallel configuration of vapor phase and liquid phase isomerization units. This configuration significantly reduces energy consumption by minimizing the amount of isomerate recycle from the vapor phase xylene isomerization and controlling the amount of C9+ aromatics that are processed in the OX and C9+ aromatics removal and subsequent OX recovery.

The invention will be better understood by reference to FIG. 2, which illustrates a specific embodiment of the invention. It will be understood by one of skill in the art that FIG. 2 is merely representative of the present invention and that many variations thereof can be readily envisioned. Moreover, various valves, compressors, and the like are not shown for convenience of view but would also be readily apparent to one of skill in the art.

As shown in FIG. 2, various feed sources comprising C8+ aromatic hydrocarbons, such as 1, 2, 15, and 17, as identified above, are sent to fractionator 16, which removes C9+ aromatics and substantially all OX from the feed. The lights are sent overhead to PX recovery 12 with intermediate removal of benzene purge 22 in fractionator 23. The overhead is sent via line 6 to PX recovery, which may be provided by a crystallization unit or selective adsorption unit (such as a Parex™ unit), per se known in the art. PX is taken off in line 7 and the PX-depleted stream comprising C8 aromatics is split and sent in parallel to vapor phase xylenes isomerization 13, having a source of hydrogen 9, and liquid phase xylene isomerization 20 via lines 30 and 40, respectively. Isomerate recycle 10 from vapor phase xylenes isomerization 13 is decreased by this process scheme and the amount of C9+ aromatics that are processed in the OX and C9+ aromatics removal 16 is better controlled.

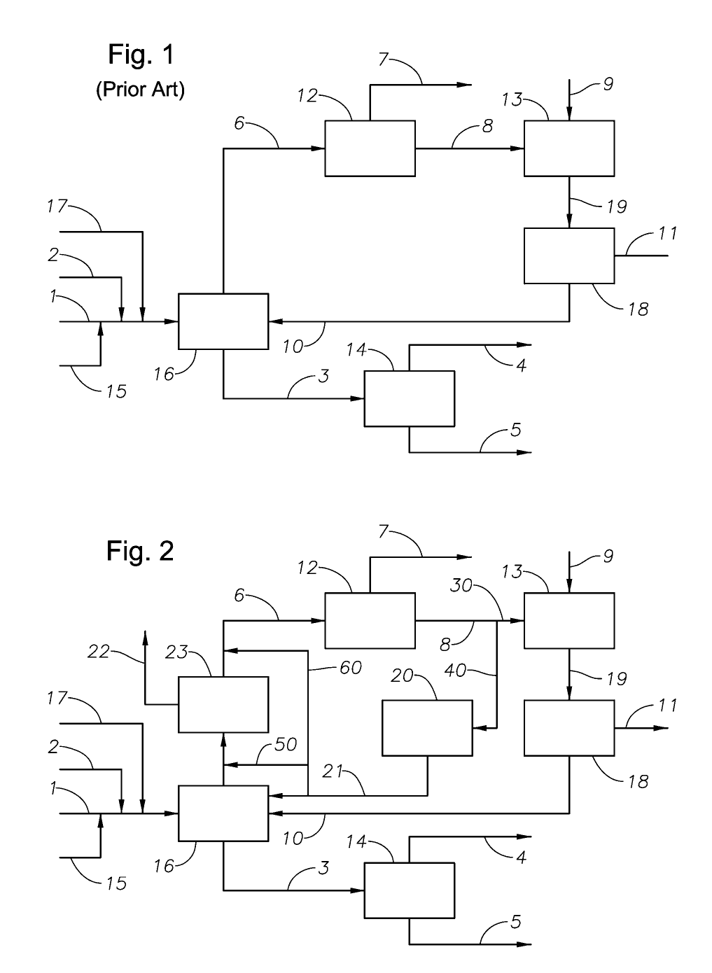

Continuing with the description of FIG. 2, the flow of PX-depleted C8 aromatics 8 is minimized through vapor phase xylenes isomerization 13 to minimize energy by reducing the amount of PX-depleted C8 aromatics stream 8 that is vaporized in vapor phase xylenes isomerization 13 and the associated amount of isomerate recycle stream 10 which contains a much higher concentration of by-product C9+ aromatics than liquid phase xylenes isomerization product 21. As in the process described for FIG. 1, in FIG. 2 the vapor phase isomerate in conduit 19 is passed through detoluenization fractionation 18, which removes C7 and lighter materials (C7−) in stream 11 to yield isomerate recycle stream is 10. Liquid phase isomerate recycle stream 21 which is the product from liquid phase xylenes isomerization 20 is sent to OX and C9+ aromatics removal 16 at a higher feed location in the column so as to minimize energy consumption due to its lower concentration of C9+ aromatics. Optionally, the product from the liquid phase isomerization can be sent via conduit 50 to benzene removal fractionator 23 as shown in FIG. 2, and/or directly back to PX recovery via conduit 60. The amount of energy savings on the OX and C9+ aromatics removal 16 and the subsequent OX fractionation 14 can result in as much as a 75% reduction in the overall energy consumption of the process for the production of PX and OX. The bottoms product 3 from fractionator 16 may be advantageously fractionated in 14 to yield an overheads product 4 of OX and bottoms product of C9+ aromatic hydrocarbons.

FIG. 2 also shows that the liquid phase isomerate recycle stream 21 can be optionally sent to one or more locations which include OX and C9+ aromatics removal 16, benzene removal 23, and directly to PX recovery 12. The amount sent to each location is determined by the need to remove by-products which include benzene, and C9+ aromatics. The by-products from liquid phase xylenes isomerization 20 in the liquid phase isomerate recycle stream 21 may need to be removed down to a level that is acceptable for PX recovery 12 especially if selective adsorption is used for recovering paraxylene. The C9+ aromatics can be removed in the OX and C9+ aromatics removal 16 or in one or more devices that employ separation techniques such as membrane, extraction, and adsorption. Similarly, benzene can be removed using one or more devices that employ separation techniques such as distillation, extraction, membrane, and adsorption. Optionally, the C9+ aromatics and benzene can be removed simultaneously using one or more devices that employ separation techniques such as distillation, extraction, membrane, and adsorption.

Regarding separation of xylenes in the PX recovery, two preferred methods are fractional crystallization and selective adsorption, the details of which are per se known in the art. See, for instance, in this regard, example U.S. Pat. No. 7,439,412, and also references cited in the Background section above. The details of crystallization and selective adsorption are not per se the subject of the present invention.

Likewise, the details of vapor phase xylenes isomerization and liquid phase xylenes isomerication are also per se known in the art. In this regard, see for example, U.S. Pat. Nos. 6,180,550; 6,448,459; 6,872,866; 7,244,409; 7,371,913; 7,495,137; 7,592,499; U.S. Patent Application Publication No. 2009-0182182; U.S. application Ser. No. 12/612,007, and Provisional Patent Application No. 61/326,445, filed Apr. 21, 2010.

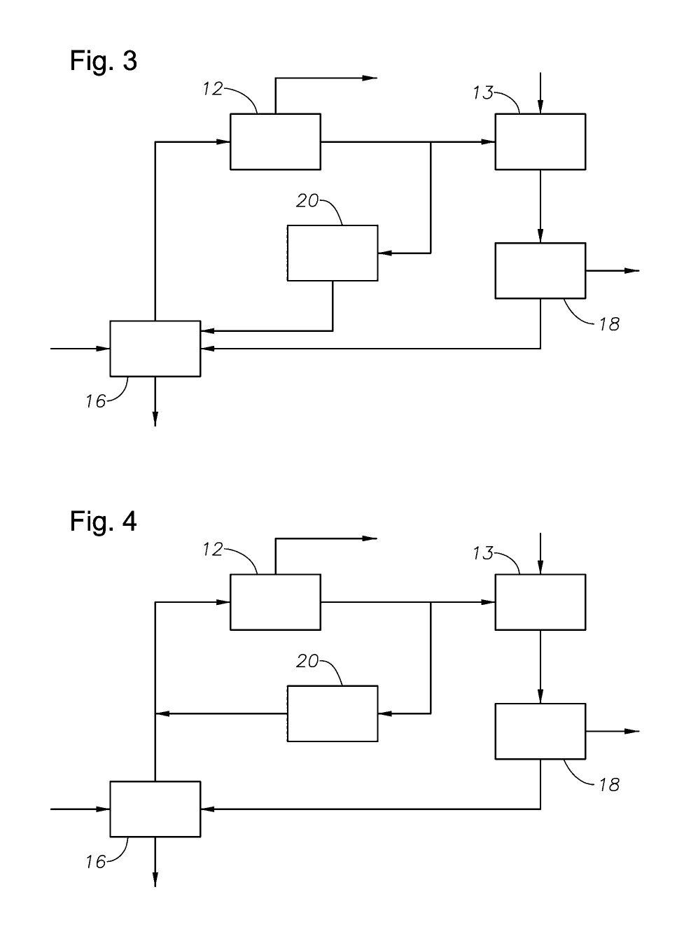

Computer simulations using the Pro II program, commercially available, were conducted to verify the benefits of the present invention. Certain assumptions were made, within the skill of the ordinary artisan in possession of the present disclosure. See, for instance, U.S. Pat. No. 7,439,412. A plant with a PX capacity of 540 kta served as the base case (FIG. 1). The simulations studied two process arrangements: Process A shown in FIG. 3, and Process B shown in FIG. 4. In both Processes A and B, the PX-depleted stream of C8 aromatics from PX recovery unit 12 was split into two equal fractions, one of which was sent to a liquid phase xylene isomerization unit 13, while the other to a vapor phase xylene isomerization unit 20. As shown in the figures, Process A (FIG. 3) sent the product from the liquid phase isomerization unit 20 to the rerun tower 16, while Process B (FIG. 4) sent the product to the Parex unit 12. In both cases, the vapor phase isomerate from vapor phase isomerization unit 13 is passed through detoluenization fractionator 18 in the same manner and to the same effect as in FIGS. 1 and 2, and the equilibrium or near equilibrium xylene isomerate sent back to the rerun tower 16. The simulations show that using the process according to the invention, there are significant energy savings of 13.10 MW (mega Watts) for Process B (FIG. 4), and 12.45 MW for Process A (FIG. 3), compared to the base case of FIG. 1.

While the illustrative embodiments of the invention have been described with particularity, it will be understood that various other modifications will be apparent to, and can be readily made by those skilled in the art, without departing from the spirit and scope of the invention.

In another embodiment, this invention relates to:

1. A process for producing paraxylene comprising:

(a) separating a feed comprising C8+ aromatics in a first fractionation step into a first stream comprising paraxylene (PX) and metaxylene (MX), and a second stream comprising orthoxylene (OX) and C9+ aromatics;

(b) separating said first stream in a PX recovery unit to recover a PX-rich stream and a PX-depleted stream;

(c) passing a first portion of said PX depleted stream through a vapor phase xylenes isomerization unit and a second portion of said PX-depleted stream through a liquid phase xylenes isomerization unit; and

(d) separating said second stream into a stream comprising OX and a stream comprising C9+ aromatics.

2. The process of paragraph 1, including at least one benzene separation step selected from a benzene separation step between said liquid phase xylenes isomerization and said first fractionation step, and a benzene separation step between the liquid phase isomerization unit and the PX recovery unit.
3. The process of one of paragraphs 1 and 2, wherein said liquid phase isomerization product is recycled to one or more of said first fractionation step, said benzene separation step (where present) and said PX recovery step.
4. The process of any one of the preceding paragraphs, wherein said liquid phase isomerization product is recycled to said first fractionation step.
5. The process of any one of paragraphs 2 and 3, wherein said liquid phase isomerization product is recycled to said benzene separation step.
6. The process of any one of the preceding paragraphs, wherein said liquid phase isomerization product is recycled to said PX recovery step.
7. The process of any one of the preceding paragraphs, wherein said PX recovery step includes a crystallization unit.
8. The process of any one of the preceding paragraphs, wherein said PX recovery step includes selective adsorption.
9. The process of any one of the preceding paragraphs, further including a step of fractionation of an isomerate recycle stream to remove toluene, if present in said stream.
10. The process of any one of the preceding paragraphs, wherein said feed comprising C8+ aromatics includes at least one feed selected from the group consisting of a C8+ selective toluene disproportionation product, a C8+ transalkyation product, a C8+ reformate product, and a C8+ toluene disproportionation product.
11. In an apparatus for the production of paraxylene (PX) and orthoxylene (OX) comprising a first fractionation column operating at conditions suitable for the separation of a C8+ aromatics stream into an overheads comprising PX and metaxylene (MX) and a bottoms product stream comprising OX and C9+ aromatics, the overheads stream fluidly connected with a PX recovery unit, wherein said PX recovery unit provides a PX-enriched stream and a PX-depleted stream, the improvement comprising dividing a conduit carrying said PX-depleted stream so that a portion of said PX-depleted stream is passed to a vapor phase isomerization unit and another portion of said PX-depleted stream is passed to a liquid phase isomerization unit.
12. The apparatus of paragraph 11, wherein said liquid phase isomerization unit is fluidly connected so as to provide liquid phase isomerate recycle to said first fractionation column and/or to said PX recovery unit.
13. The apparatus of any one of paragraphs 11-12, wherein said PX recovery unit is selected from at least one of a crystallizer and an adsorptive separator.
14. The apparatus of any one of paragraphs 11-13, further including at least one other fractionator upstream of said first fractionator, wherein said at least one other fractionator operates under conditions suitable for removing benzene from a stream comprising xylenes or for removing toluene from a stream comprising xylenes, and optionally wherein both said fractionator for removing benzene and said fractionator for removing toluene are provided upstream of said first fractionator.
15. The apparatus of any one of paragraphs 11-14, further including at least one separation unit downstream of said first fractionation column fluidly connected to said bottoms product stream, whereby OX is separated from C9+ aromatics, said at least one separation unit selected from a crystallizer, a membrane unit, or a fractionation column.

Trade names used herein are indicated by a ™ symbol or ® symbol, indicating that the names may be protected by certain trademark rights, e.g., they may be registered trademarks in various jurisdictions. All patents and patent applications, test procedures (such as priority documents, ASTM methods, UL methods, and the like), and other documents cited herein are fully incorporated by reference to the extent such disclosure is not inconsistent with this invention and for all jurisdictions in which such incorporation is permitted. When numerical lower limits and numerical upper limits are listed herein, ranges from any lower limit to any upper limit are contemplated.

What is claimed is:

1. A process for producing paraxylene comprising:
(a) separating a feed comprising C8+ aromatics in a first fractionation step into a first stream comprising paraxylene, PX, and metaxylene, MX, and a second stream comprising orthoxylene (OX) and C9+ aromatics;
(b) separating said first stream in a PX recovery unit to recover a PX-rich stream and a PX-depleted stream;
(c) passing a first portion of said PX depleted stream through a vapor phase xylenes isomerization unit and a second portion of said PX-depleted stream through a liquid phase xylenes isomerization unit; and
(d) separating said second stream into a stream comprising OX and a stream comprising C9+ aromatics.

2. The process of claim 1, including at least one benzene separation step selected from a benzene separation step between said liquid phase xylenes isomerization and said first fractionation step, and a benzene separation step between the liquid phase isomerization unit and the PX recovery unit.

3. The process of claim 1, wherein said liquid phase isomerization product is recycled to one or more of said first fractionation step, said benzene separation step (where present) and said PX recovery step.

4. The process of claim 2, wherein said liquid phase isomerization product is recycled to one or more of said first fractionation step, said benzene separation step (where present) and said PX recovery step.

5. The process of claim 1, wherein said liquid phase isomerization product is recycled to said first fractionation step.

6. The process of claim 2, wherein said liquid phase isomerization product is recycled to said first fractionation step.

7. The process of any one of claim 1, wherein said liquid phase isomerization product is recycled to said benzene separation step.

8. The process of any one of claim 2, wherein said liquid phase isomerization product is recycled to said benzene separation step.

9. The process of claim 1, wherein said liquid phase isomerization product is recycled to said PX recovery step.

10. The process of claim 2, wherein said liquid phase isomerization product is recycled to said PX recovery step.

11. The process of claim 1, wherein said PX recovery step includes a crystallization unit.

12. The process of claim 2, wherein said PX recovery step includes a crystallization unit.

13. The process of claim 3, wherein said PX recovery step includes a crystallization unit.

14. The process of claim 1, wherein said PX recovery step includes selective adsorption.

15. The process of claim 2, wherein said PX recovery step includes selective adsorption.

16. The process of claim 1, further including a step of fractionation of an isomerate recycle stream to remove toluene, if present in said stream.

17. The process of claim 2, further including a step of fractionation of an isomerate recycle stream to remove toluene, if present in said stream.

18. The process of claim 1, wherein said feed comprising C8+ aromatics includes at least one feed selected from the group consisting of a C8+ selective toluene disproportionation product, a C8+ transalkyation product, a C8+ reformate product, and a C8+ toluene disproportionation product.

19. The process of claim 2, wherein said feed comprising C8+ aromatics includes at least one feed selected from the group consisting of a C8+ selective toluene disproportionation product, a C8+ transalkyation product, a C8+ reformate product, and a C8+ toluene disproportionation product.

20. In an apparatus for the production of paraxylene (PX) and orthoxylene (OX) comprising a first fractionation column operating at conditions suitable for the separation of a C8+ aromatics stream into an overheads comprising PX and metaxylene (MX) and a bottoms product stream comprising OX and C9+ aromatics, the overheads stream fluidly connected with a PX recovery unit, wherein said PX recovery unit provides a PX-enriched stream and a PX-depleted stream, the improvement comprising dividing a conduit carrying said PX-depleted stream so that a portion of said PX-depleted stream is passed to a vapor phase isomerization unit and another portion of said PX-depleted stream is passed to a liquid phase isomerization unit.

21. The apparatus of claim 20, wherein said liquid phase isomerization unit is fluidly connected so as to provide liquid phase isomerate recycle to said first fractionation column and/or to said PX recovery unit.

22. The apparatus of claim 20, wherein said PX recovery unit is selected from at least one of a crystallizer and an adsorptive separator.

23. The apparatus of claim 47, further including at least one other fractionator upstream of said first fractionator, wherein said at least one other fractionator operates under conditions suitable for removing benzene from a stream comprising xylenes or for removing toluene from a stream comprising xylenes, and optionally wherein both said fractionator for removing benzene and said fractionator for removing toluene are provided upstream of said first fractionator.

24. The apparatus of claim 20, further including at least one separation unit downstream of said first fractionation column fluidly connected to said bottoms product stream, whereby OX is separated from C9+ aromatics, said at least one separation unit selected from a crystallizer, a membrane unit, or a fractionation column.

25. The apparatus of claim 20, further including at least one separation unit downstream of said first fractionation column fluidly connected to said bottoms product stream, whereby OX is separated from C9+ aromatics, said at least one separation unit selected from a crystallizer, a membrane unit, or a fractionation column.
